(19) United States
(12) Patent Application Publication
(10) Pub. No.: US 2013/0334851 A1
(43) Pub. Date: Dec. 19, 2013

(54) KIOSK WITH SEAT ASSEMBLY

(71) Applicant: Solohealth, Inc., Duluth, GA (US)

(72) Inventors: ERIC W. HOELL, SUGAR HILL, GA (US); STEVEN J. DUERO, WAUBEKA, WI (US)

(73) Assignee: Solohealth, Inc., Duluth, GA (US)

(21) Appl. No.: 13/905,179

(22) Filed: May 30, 2013

Related U.S. Application Data
(60) Provisional application No. 61/656,898, filed on Jun. 7, 2012.

(51) Int. Cl. A47C 3/18; A61B 3/00; A47C 7/62

(52) U.S. Cl. CPC A47C 3/18; A47C 7/62; A61B 3/00
USPC 297/217.2; 297/344.21; 297/344.22; 600/558

(57) ABSTRACT

A kiosk may include a main body with a front surface and a seat assembly located near the main body adjacent to the front surface of the main body.

### CROSS-REFERENCE TO RELATED APPLICATIONS

This application is based on and derives the benefit of the filing date of U.S. Provisional Patent Application No. 61/656,898, filed Jun. 7, 2012. The entire content of this application is herein incorporated by reference in its entirety.

### BRIEF DESCRIPTION OF THE DRAWINGS

FIG. 1A is a perspective view of a kiosk according to a first embodiment of the invention.

FIG. 1B is a perspective view of a kiosk according to the first embodiment of the invention.

FIG. 1C is a perspective view of a kiosk according to a second embodiment of the invention.

FIG. 1D is a perspective view of a kiosk according to the second embodiment of the invention.

FIG. 2A is a rear elevation view of a rotatable seat assembly according to the first embodiment of the invention.

FIG. 2B is a front elevation view of a rotatable seat assembly according to the first embodiment of the invention.

FIG. 2C is a side elevation view of a rotatable seat assembly according to the first embodiment of the invention.

FIG. 2D is a side elevation view of a rotatable seat assembly according to the first embodiment of the invention.

FIG. 2E is a rear elevation view of a rotatable seat assembly according to the second embodiment of the invention.

FIG. 2F is a front elevation view of a rotatable seat assembly according to the second embodiment of the invention.

FIG. 2G is a side elevation view of a rotatable seat assembly according to the second embodiment of the invention.

FIG. 2H is a side elevation view of a rotatable seat assembly according to the second embodiment of the invention.

FIG. 3A is a cutaway rear elevation view of a rotatable seat assembly according to the second embodiment of the invention.

FIG. 3B is a rear elevation view of a latch assembly according to the second embodiment of the invention.

FIG. 3C is a cutaway rear elevation view of a rotatable seat assembly according to the second embodiment of the invention.

FIG. 3D is a rear elevation view of a seat post according to the second embodiment of the invention.

FIG. 3E is a cutaway front elevation view of a rotatable seat assembly according to the second embodiment of the invention.

FIG. 4A is a perspective view of a kiosk according to a third embodiment of the invention.

FIG. 4B is a perspective view of a kiosk according to a fourth embodiment of the invention.

FIG. 5A is a rear elevation view of a fixed seat assembly according to the third embodiment of the invention.

FIG. 5B is a front elevation view of a fixed seat assembly according to the third embodiment of the invention.

FIG. 5C is a side elevation view of a fixed seat assembly according to the third embodiment of the invention.

FIG. 5D is a side elevation view of a fixed seat assembly according to the third embodiment of the invention.

FIG. 6 is a cutaway front elevation view of a fixed seat assembly according to the third embodiment of the invention.

### DETAILED DESCRIPTION OF SEVERAL EMBODIMENTS

Kiosks which may be used to generate information and/or present information to a user are described herein. In some examples, kiosks may be used for health care screening. Kiosks may present general health care information to users and/or may gather information about users to generate and present personalized information for individual users. Health care kiosks may include diagnostic devices such as blood pressure cuffs and/or scales. Some kiosk embodiments described herein may include a seat assembly with a built in scale which may be used to measure the weight of a user seated on a seat that is part of the seat assembly. Some kiosk embodiments described herein may include a seat assembly (which may or may not include a scale) which may be moved from a position in front of a kiosk screen and/or interface to a stowed position. This may allow a user in a wheelchair to use the kiosk, for example.

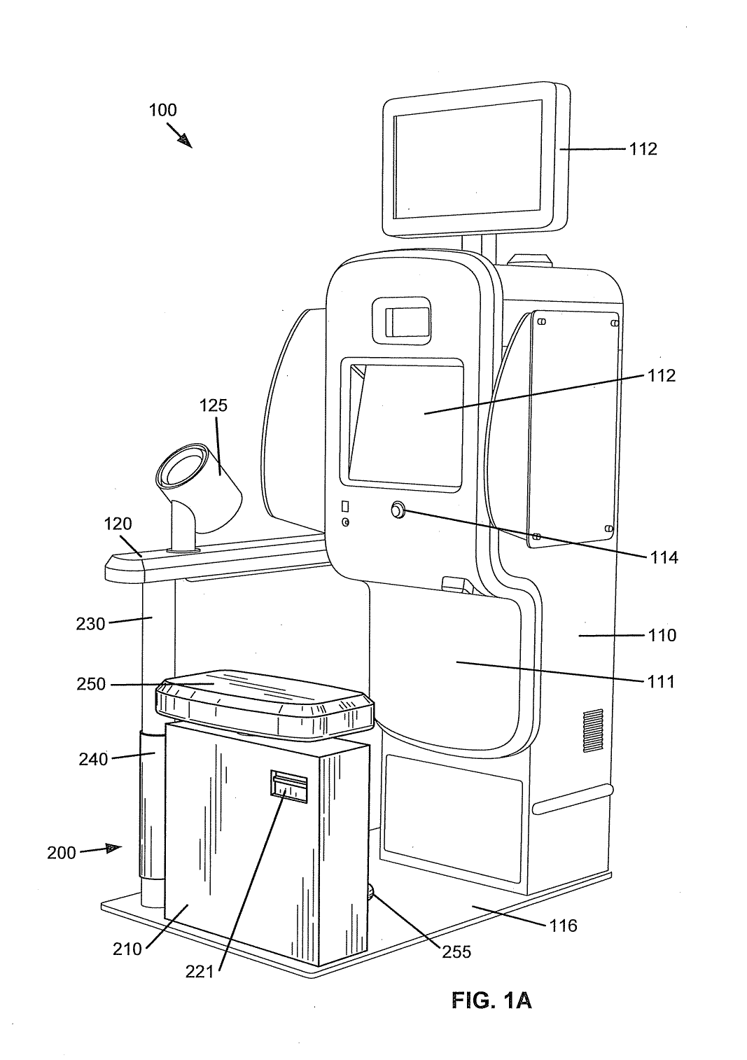

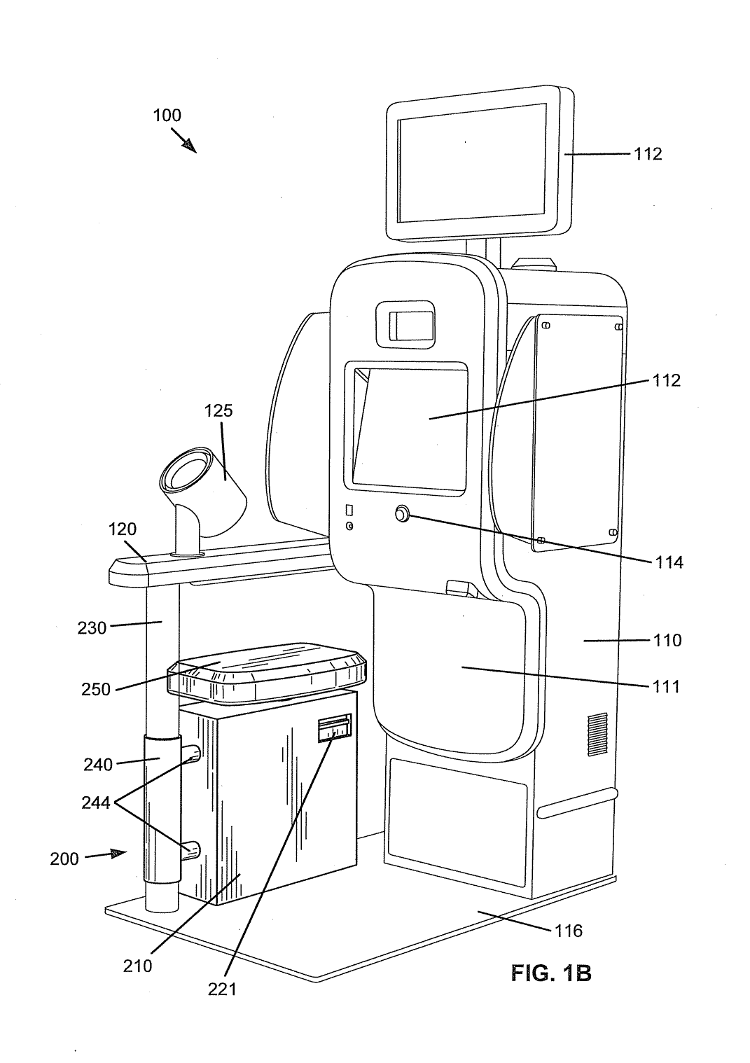

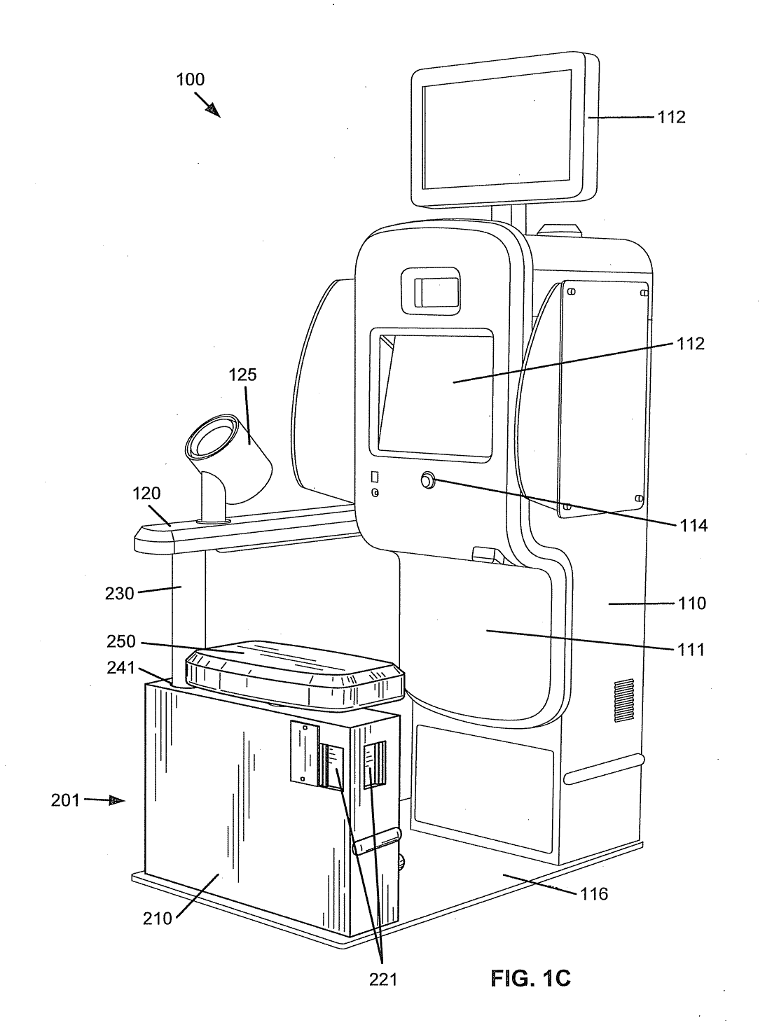

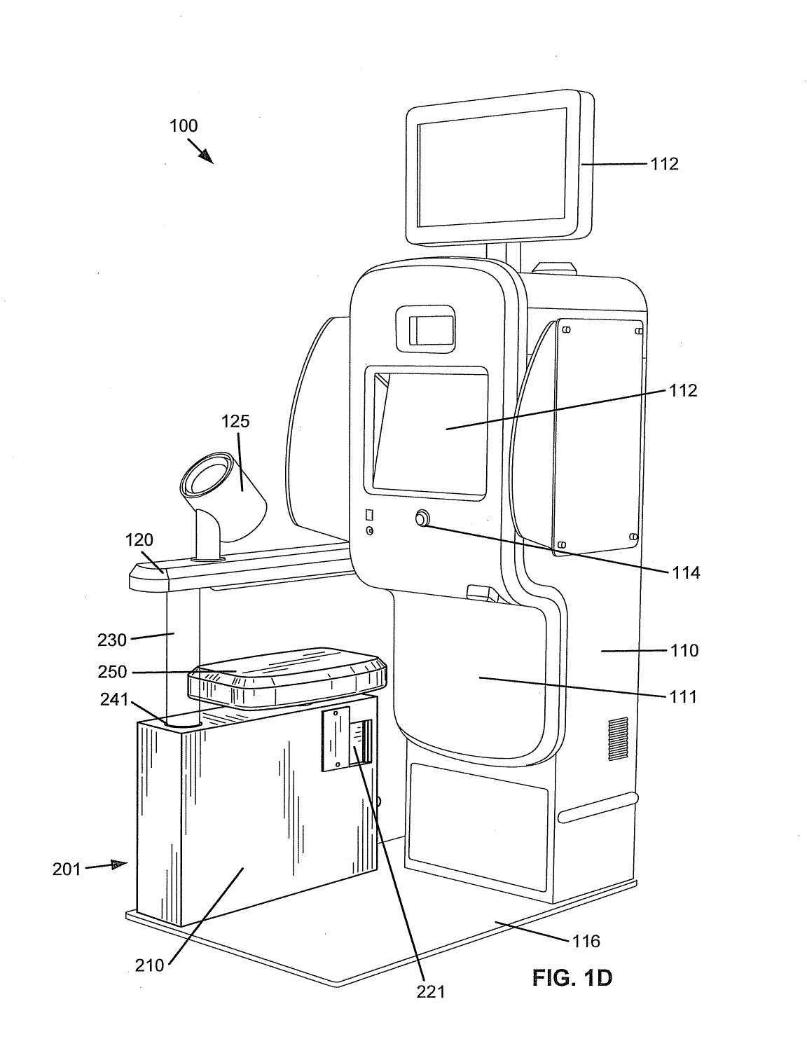

FIG. 1A is a perspective view of a kiosk 100 according to an embodiment of the invention. The kiosk 100 may include a body 110 which may house and/or support other components of the kiosk. For example, one or more displays 112 may be mounted in or on the body 110. These displays 112 may be screens for displaying text, graphics, and/or video; speakers and/or headphone jacks for presenting audio; and/or any other type of display devices. One or more input devices 114 may also be mounted on the body. Input devices 114 may enable a user of the kiosk 100 to enter information and/or commands. Input devices 114 may include buttons, keyboards, touch screen displays 112, barcode scanners, magnetic card swipe devices, and/or any other type of input device. In some embodiments, the kiosk 100 may include a base plate 116 which may be attached to or integrated with the body 110. In other embodiments, the body 110 may be located on a pre-existing floor surface without a base plate 116. The body 110 may have a front surface 111 that may be intended for user interaction. Therefore, components such as the displays 112 and/or input devices 114 may be mounted in or on the front surface 111.

The kiosk 100 may include an arm rest 120, which may be attached or positioned adjacent to the body 110. In some embodiments, a diagnostic tool 125, such as a blood pressure cuff may be mounted on the arm rest 120. Diagnostic tools 125 may alternatively or additionally be mounted on other parts of the kiosk 100, such as the body 110. For example, a mirror box which may be used to test vision may be mounted in the body 110. Diagnostic tools 125 may be used to detect and/or measure a user's health related information, such as the user's blood pressure, pulse, body mass index, vision, and/or other information. The arm rest 120 may be supported by a post (such as a seat post 230, described in greater detail below) in some embodiments. For example, one end of the aim rest 120 may be attached to the body 110, and the opposite end may be supported by the post.

The kiosk 100 may include one or more computers which may be housed within the body 110, for example. A computer may be any programmable machine capable of performing arithmetic and/or logical operations. In some embodiments, computers may comprise processors, memories, data storage devices, and/or other commonly known or novel components. These components may be connected physically or through network or wireless links. Computers may also comprise software which may direct the operations of the aforementioned components. Computers may be referred to with terms that are commonly used by those of ordinary skill in the relevant art, such as servers, PCs, and other terms. It will be understood by those of ordinary skill that those terms used herein are interchangeable, and any computer capable of performing the described functions may be used. In some cases, computers may be interconnected via one or more networks. A network may be any plurality of completely or partially interconnected computers wherein some or all of the computers are able to communicate with one another via wired and/or wireless connection using any of a variety of interfaces and/or protocols.

A computer within the kiosk body 110 may include a processor in communication with the display 112, input device 114, diagnostic device 125, and/or load sensor 260. The processor may receive data from the diagnostic device 125 and/or load sensor 260 (described in greater detail below) and process this data to generate information which may be of interest to the user, such as health information and/or recommendations. The processor may cause the display 112 to display the generated information. The processor may also receive user commands and/or information from the input device 114, may perform processing in response to the received commands and/or information, and may cause the display 112 to display information based on this processing. In some embodiments, data ports such as universal serial bus (USB) ports may be provided on the body 110 to enable a user to retrieve data generated and/or measured by the computer.

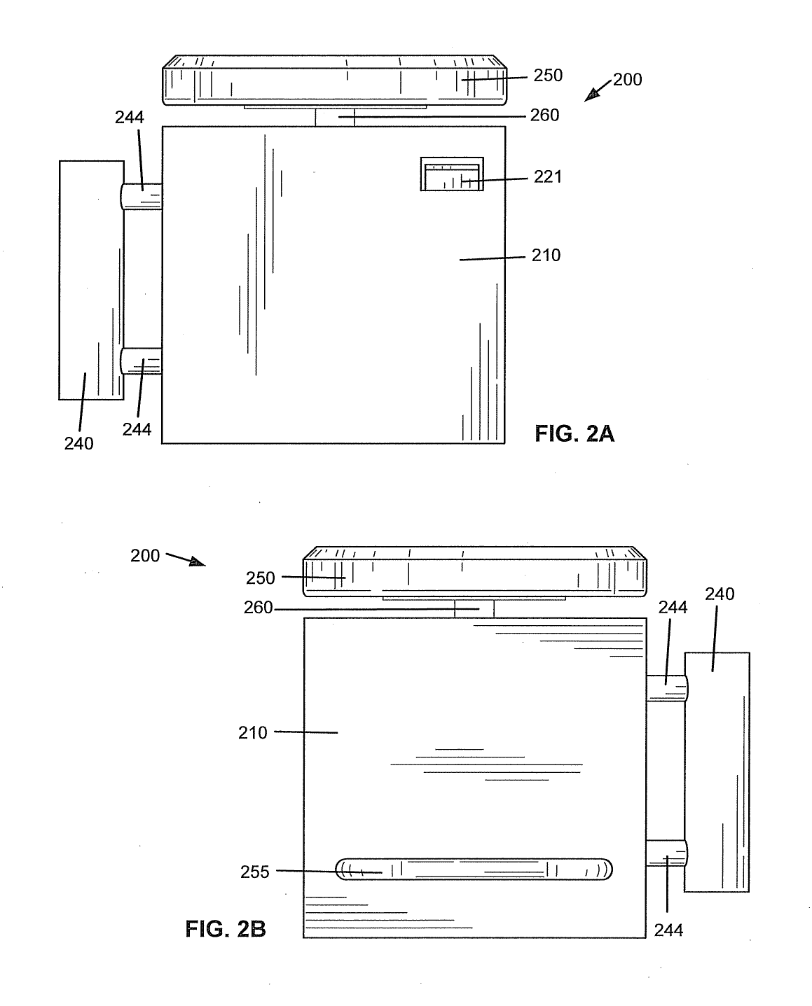

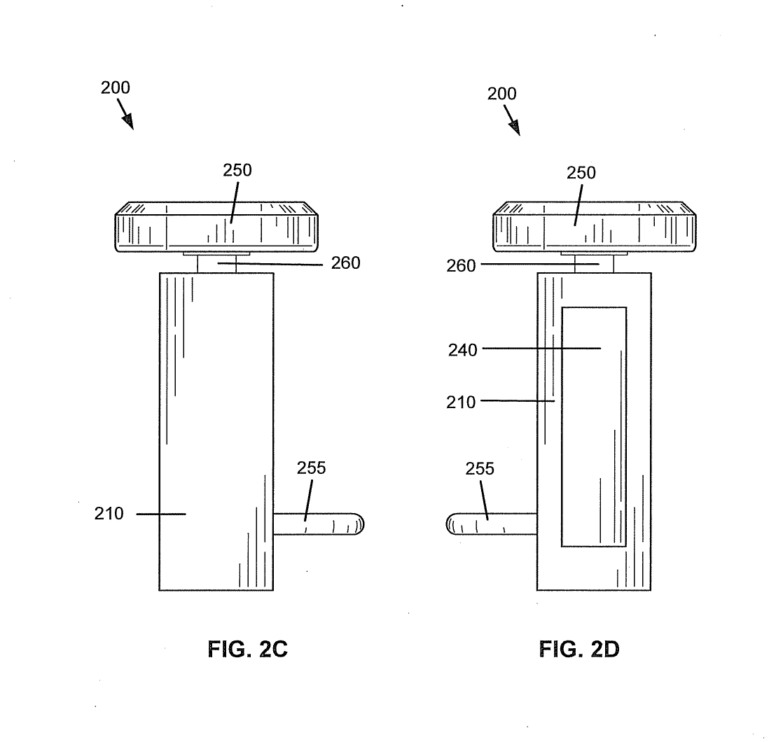

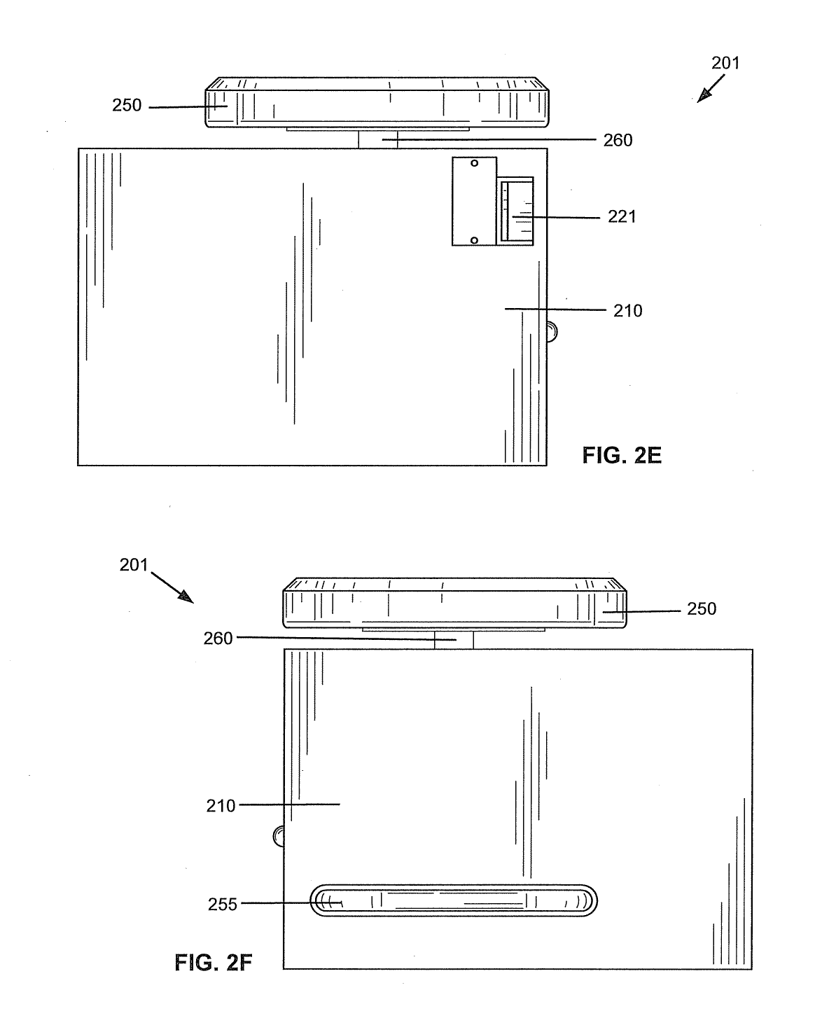

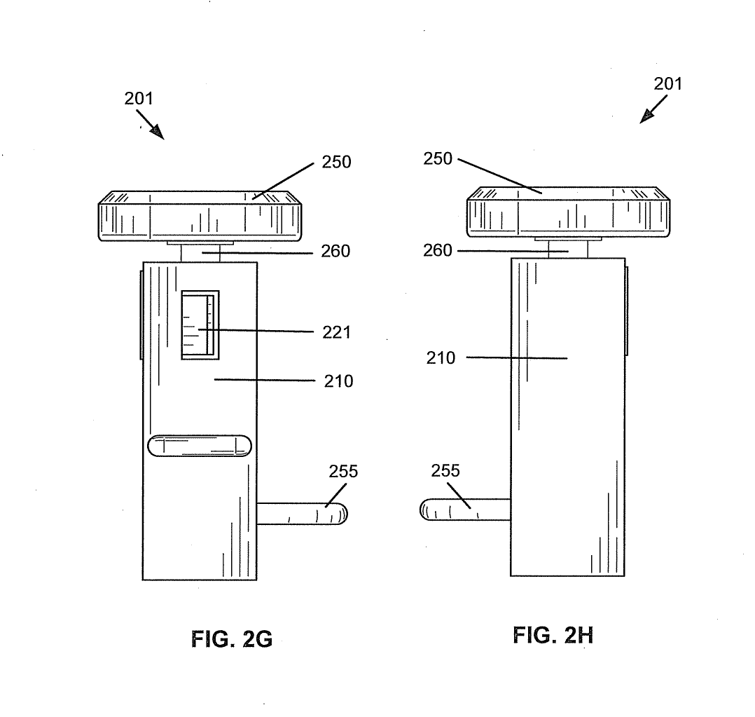

The kiosk 100 may also include a rotatable seat assembly 200. FIGS. 2A-2D are elevation views of a rotatable seat assembly 200 according to an embodiment of the invention. The seat assembly 200 may include a seat support 210 and a seat 250 disposed above the seat support 210. In the example of FIGS. 2A-2D, the seat support 210 is a rectangular box which may contain internal parts described in greater detail below, but the seat support 210 may be any shape or configuration capable of supporting a seat 250.

The seat support 210 may be rotatably coupled to a vertical seat post 230 so that the seat assembly 200 may be rotated about the seat post 230. FIG. 1B is a perspective view of a kiosk 100 according to an embodiment of the invention wherein the seat assembly 200 has been rotated approximately ninety degrees from the position shown in FIG. 1A. The seat support 210 may be rotatably coupled to the seat post 230 with a hinge assembly 240 so that the seat may be rotated into a plurality of positions. In the example of FIGS. 2A-2D, the hinge assembly 240 may include a barrel 242 surrounding the seat post 230 and attached to the seat support 210 with one or more connecting shafts 244. In the example of FIGS. 2E-2H, the rotatable seat assembly 201 may include a hinge assembly 241 which may be an assembly which defines a space in the seat support 210 into which the seat post 230 may be inserted so that the seat support 210 surrounds the seat post 230. For example, the space may be defined by a hole in a top surface of the seat support 210 and a hole in a bottom surface of the seat support 210 and/or by a hollow tube in the seat support 210 into which the seat post 230 may be inserted. FIG. 1C is a perspective view of a kiosk 100 according to an embodiment of the invention wherein the rotatable seat assembly 201 and hinge assembly 241 of FIGS. 2E-2H is used. FIG. 1D is a perspective view of a kiosk 100 according to an embodiment of the invention wherein the seat assembly 201 has been rotated approximately ninety degrees from the position shown in FIG. 1C. Either of these configurations may allow the seat post 230 to define the axis of rotation for the seat support 210. Other hinge assembly 240/241 configurations may also be used.

One or more handles 221 or other controls may be disposed on one or more exterior surfaces of the seat support 210. As described in greater detail below, the handles 221 may be used to operate a latch mechanism 220 which may selectively engage the seat assembly 200/201 with the seat post 230 and/or a base plate 116 or other floor surface not integral to the kiosk 100.

A seat 250 may be disposed on the top side of the seat support 210. In some embodiments, the seat 250 may be coupled to the seat support 210 with a load sensor 260, which is described in greater detail below. The load sensor 260 may be used to measure the weight of a user sitting on the seat 250. A foot rest 255 may be attached to or integrated with the seat 250 so that weight on the foot rest 255 may be applied to the load sensor 260.

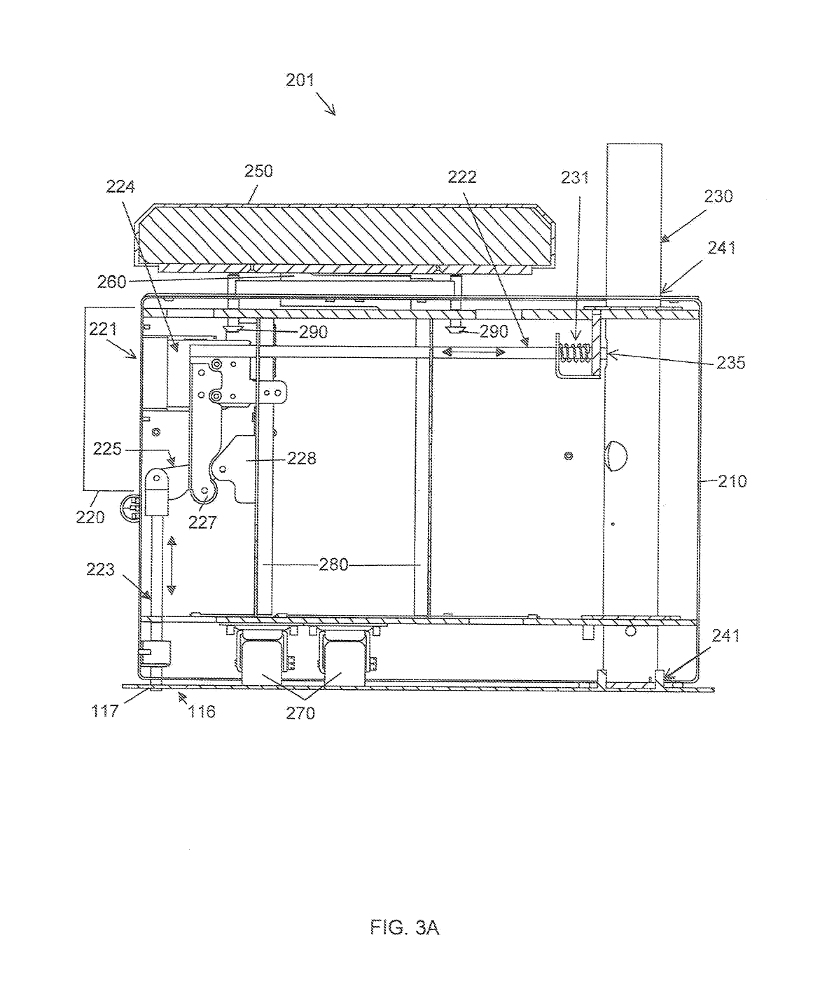

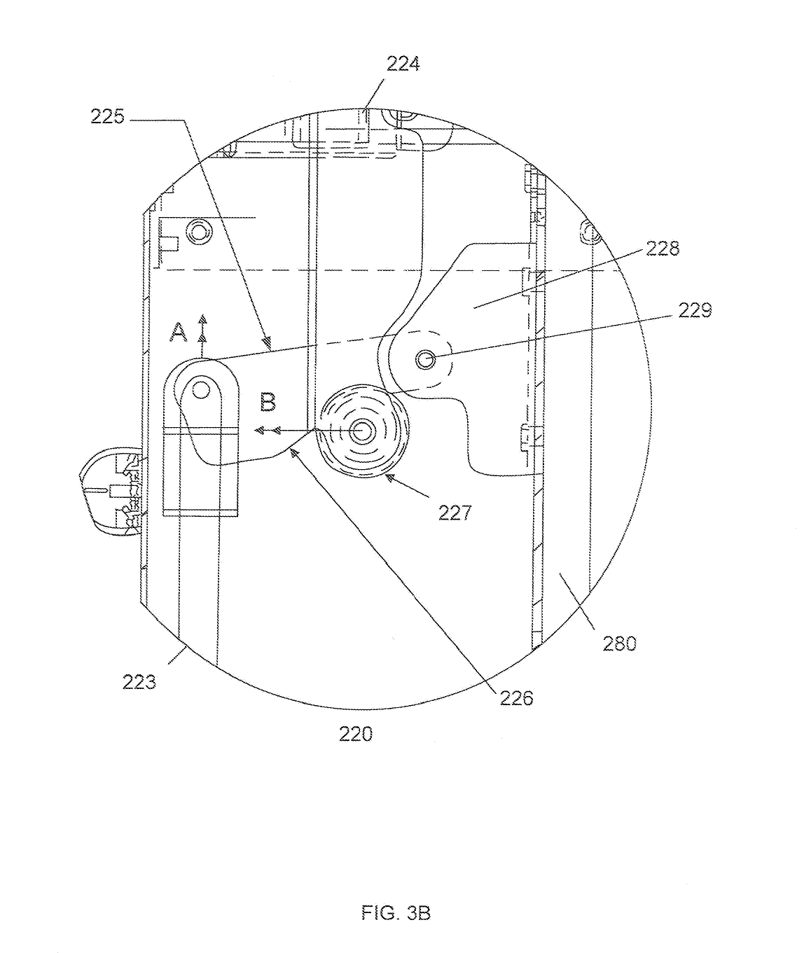

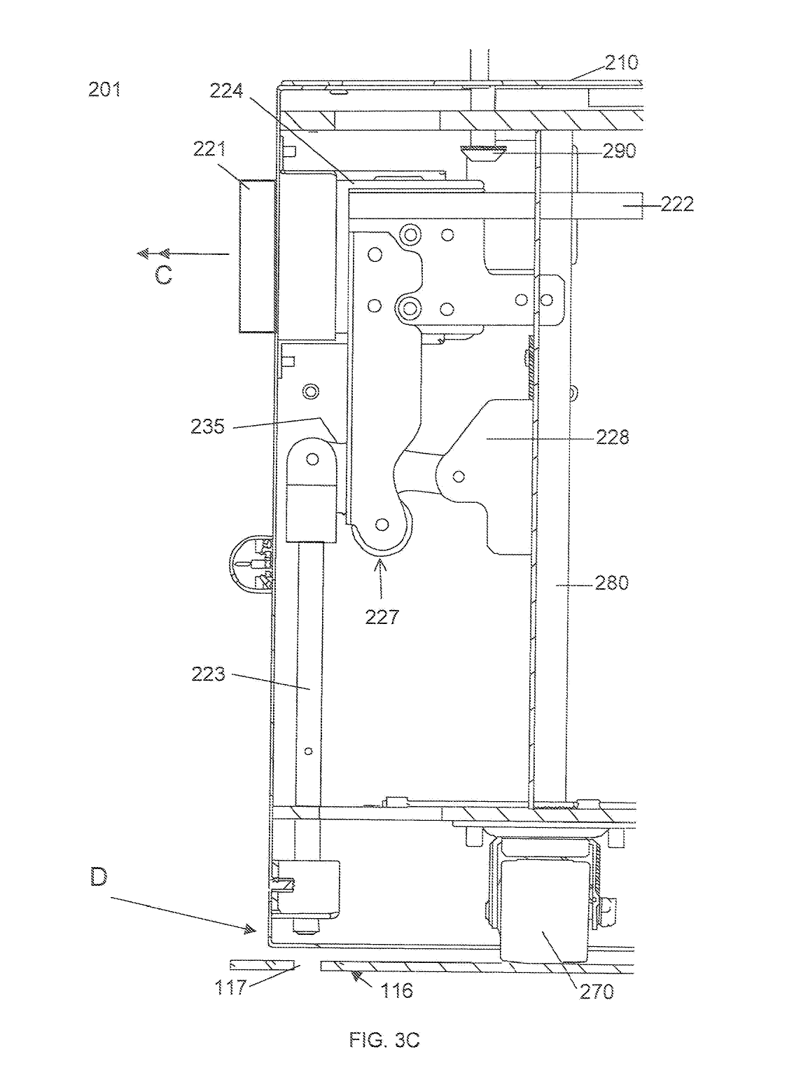

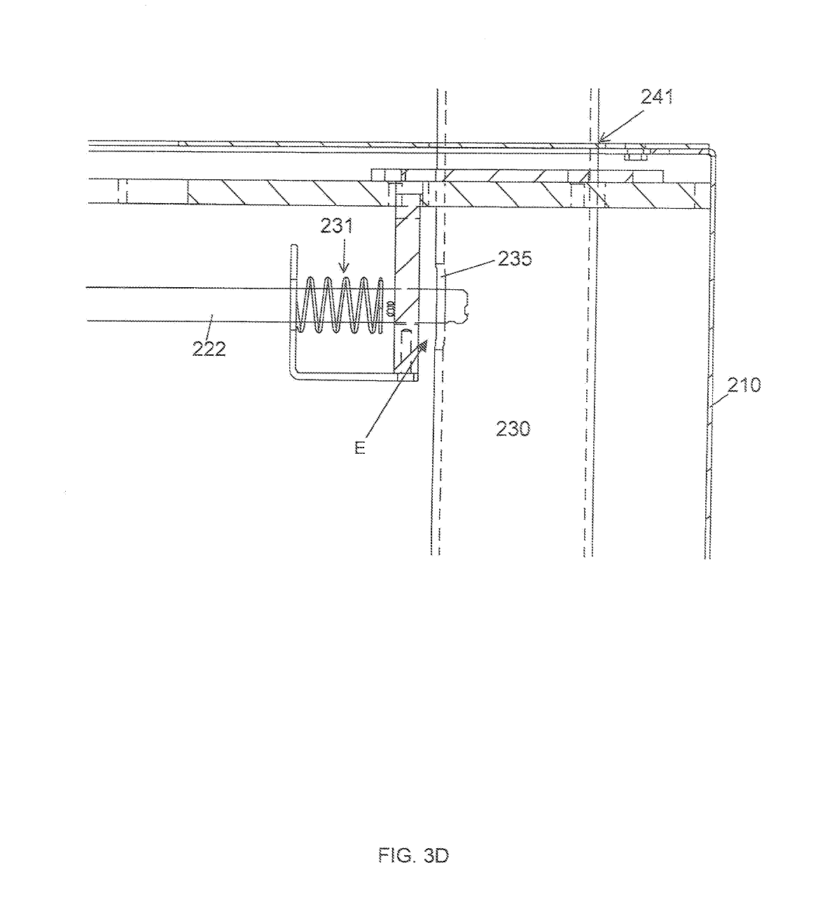

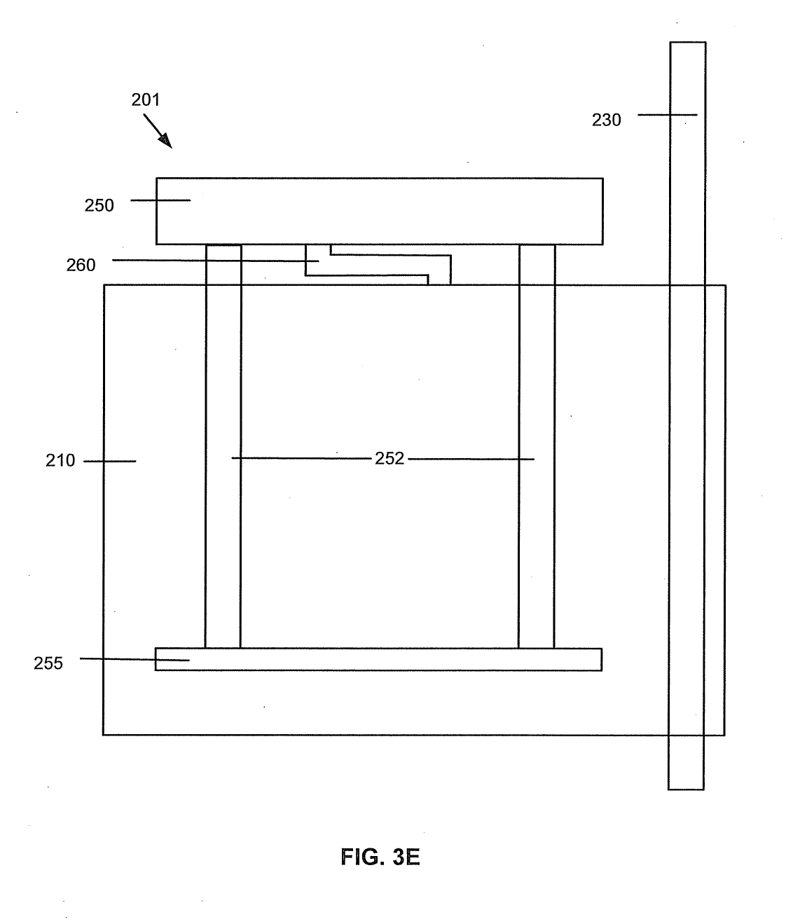

FIG. 3A is a cutaway rear elevation view of the rotatable seat assembly 201 according to an embodiment of the invention. Some embodiments of the seat assembly 200/201 may include a substantially hollow seat support 210 which may contain various components such as internal frame elements 280 and other components. For example, one or more casters 270 may be disposed at the bottom of the seat support 210 so that the seat support 210 may be rolled over a base plate 116 or other floor surface. For example, if the seat assembly 200/201 is rotated, it may roll on the casters 270 while moving from one position to another. Furthermore, the casters 270 may support the weight of the seat assembly 200/201 and also the weight of a user seated on the seat 250, when applicable.

The seat support 210 may also contain a latch assembly 220. The latch assembly 220 may be used to engage the seat assembly 200/201 with the seat post 230 and/or a floor surface in at least a position substantially parallel to the front surface 111 of the kiosk body 110 and a position substantially perpendicular to the front surface 111 of the kiosk body 110 (for example, under the arm rest 120). The seat assembly 200/201 position that is substantially parallel to the front surface 111 may be a position wherein a user may sit on the seat 250 and view the displays 112, operate the controls 114, and/or use the diagnostic tools 125. The seat assembly 200/201 position that is substantially perpendicular to the front surface 111 may be a stowed position, which may be adopted to provide space for a user in a wheelchair to operate the kiosk 100, for example.

As noted above, one or more handles 221 may be included in the latch assembly 220. A handle 221 or other control may be coupled to a post rod 222 such that pulling on or otherwise actuating the handle 221 may cause the post rod 222 to be withdrawn from one of a plurality of holes 235 in the seat post 230. Releasing the handle 221 may cause the post rod 222 to be inserted into one of the plurality of holes 235 if the post rod 222 is disposed adjacent to one of the holes 235, or releasing the handle 221 may cause the post rod 222 to contact and/or rub against the seat post 230 if the post rod 222 is not disposed adjacent to one of the holes 235. In some embodiments, the post rod 222 may be partially disposed within a connecting shaft 244, and this connecting shaft 244 may be at a same height as the plurality of holes 235. In other embodiments, the post rod 222 may be disposed entirely within the seat support 210 when the handle is actuated.

For example, one of the holes 235 may be placed in a position such that inserting the post rod 222 into the hole 235 fixes the seat assembly 200/201 in the position substantially parallel to the front surface 111, and another of the holes 235 may be placed in a position such that inserting the post rod 222 into the hole 235 fixes the seat assembly 200/201 in the position substantially perpendicular to the front surface 111. A spring 231 may be coupled to the post rod 222 such that the spring 231 may provide a bias to the post rod 222 that may move the post rod 222 towards the hole 235 when the handle 221 is released. FIG. 3D is a rear elevation view of a seat post 230 according to an embodiment of the invention, showing this situation in detail. At point E, the post rod 222 may enter a hole 235 in the seat post 230.

FIG. 3B is a rear elevation view of a latch assembly 220 according to an embodiment of the invention. The handle 221 may also be coupled to an actuator plate 224. The actuator plate 224 may be configured so that, when the handle 221 is pulled or otherwise actuated, the actuator plate 224 moves laterally in a direction B. In turn, a roller 227, which may be rotatably attached to the actuator plate 224, may cause a lift cam 225 to pivot about a pivot point 229 as a cam surface 226 rides on the roller 227. The lift cam 225 may be coupled to a floor rod 223 such that the pivoting of the lift cam 225 may cause the lift cam 225 to lift the floor rod 223 in a direction A.

FIG. 3C is a cutaway rear elevation view of the rotatable seat assembly 201 according to an embodiment of the invention. As seen in FIG. 3C, when the handle 221 is actuated in direction C, the actuator plate 224 may be moved in direction C, and the floor rod 223 may be lifted. Furthermore, the actuation of the handle 221 in direction C may cause the post rod 222 to move in direction C. Actuating the handle 221 may cause the floor rod 223 to be withdrawn from one of a plurality of holes 117 in the base plate 116 or other floor surface. Releasing the handle 221 may cause the floor rod 223 to be inserted into one of the plurality of holes 117 if the floor rod 223 is disposed adjacent to one of the holes 117, as shown in FIG. 3A. One of the holes 117 may be placed in a position such that inserting the floor rod 223 into the hole 117 fixes the seat assembly 200/201 in the position substantially parallel to the front surface 111, and another of the holes 117 may be placed in a position such that inserting the floor rod 223 into the hole 117 fixes the seat assembly 200/201 in the position substantially perpendicular to the front surface 111. Therefore, if the handle 221 is released when the seat assembly 200/201 is in one of the positions wherein it can be fixed, the post rod 222 may enter a hole 235 and the floor rod 223 may enter a hole 117.

In some situations, the handle 221 may be actuated and the seat assembly 200/201 may be moved to a position wherein the post rod 222 is not adjacent to a hole 235. If the handle 221 is released when the seat assembly 200/201 is in this position, the actuator plate 224 and post rod 222 may move in a direction opposite the C direction until the post rod 222 makes contact with the seat post 230. The respective lengths of the post rod 222 and the floor rod 223 may be such that, in this situation, the floor rod 223 is stopped before making contact with the base plate 116 or other floor surface. This may prevent scraping of the base plate 116 or other floor surface by the floor rod 223 while the seat assembly 200/201 is being rotated.

FIG. 3E is a cutaway front elevation view of a rotatable seat assembly 201 according to an embodiment of the invention. As noted above, in some embodiments the seat 250 may be coupled to the seat support 210 with a load sensor 260. The load sensor 260 may be partially contained within the seat support 210. In one example, the load sensor 260 may be a load cell with one end attached to the seat 250 and another end attached to the seat support 210, although other load sensor types may be used instead of or in addition to the load cell. When a load, for example a user, is placed on the seat, the load sensor 260 may detect the load. For example, the load sensor may enable the computer of the kiosk 100 to determine and/or report a user's weight and/or body mass index. The foot rest 255 may be attached to the seat 250 so that the foot rest 255 is supported by the seat 250. Weight on the foot rest 255 may therefore be applied to the load sensor 260. For example, a user may place their feet on the foot rest 255 so that their entire weight may be measured by the load sensor 260. In some embodiments, such as the example of FIG. 3E, a portion of the foot rest 255 may be contained within the seat support 210, with a portion of the foot rest 255 useful for supporting a user's feet protruding from an opening in the seat support 210. For example, the foot rest 255 may include vertical connecting elements 252 which may connect the foot rest 255 with the seat 250. These vertical connecting elements 252 may be inside the seat support 210. One or more bolts 290 may be disposed on the top of the seat support 210 and may extend to a position beneath the seat 250 without making contact with the seat 250. Therefore, if the load sensor 260 is deformed, for example because an excessive or unexpected load is applied to the seat 250, the weight of the seat 250 and the load on the seat 250 may be supported by the bolts 290 (and therefore the seat support 210 and casters 270).

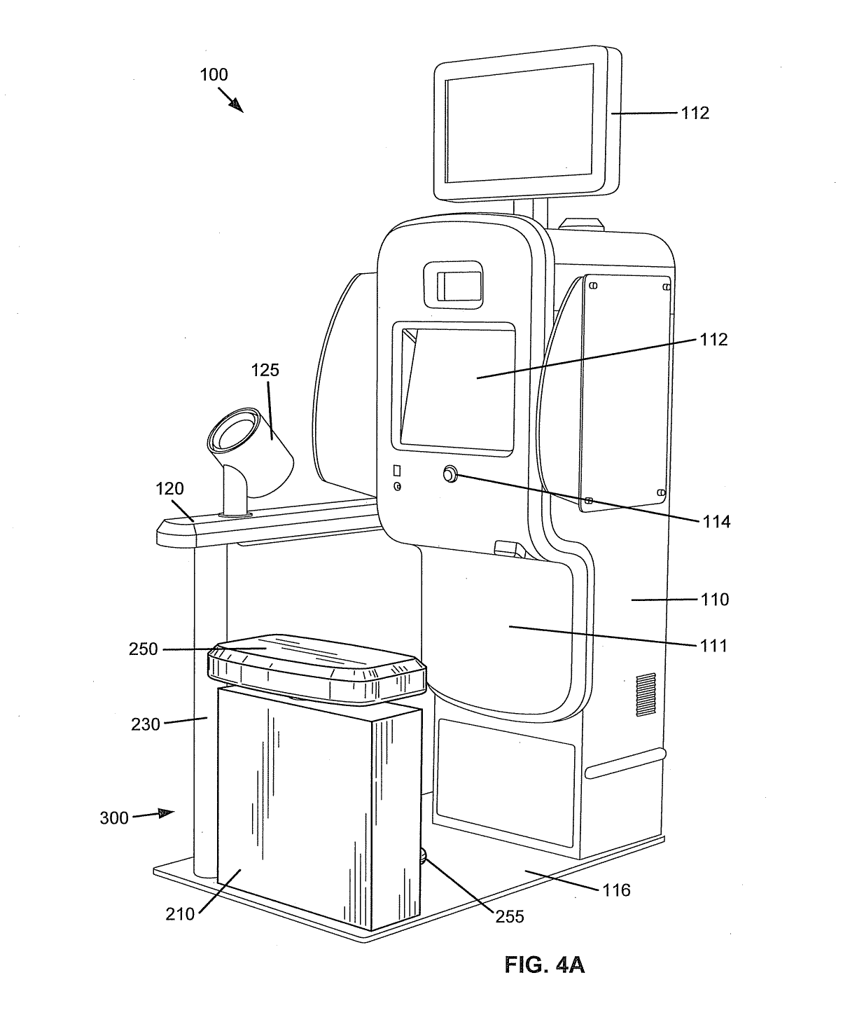

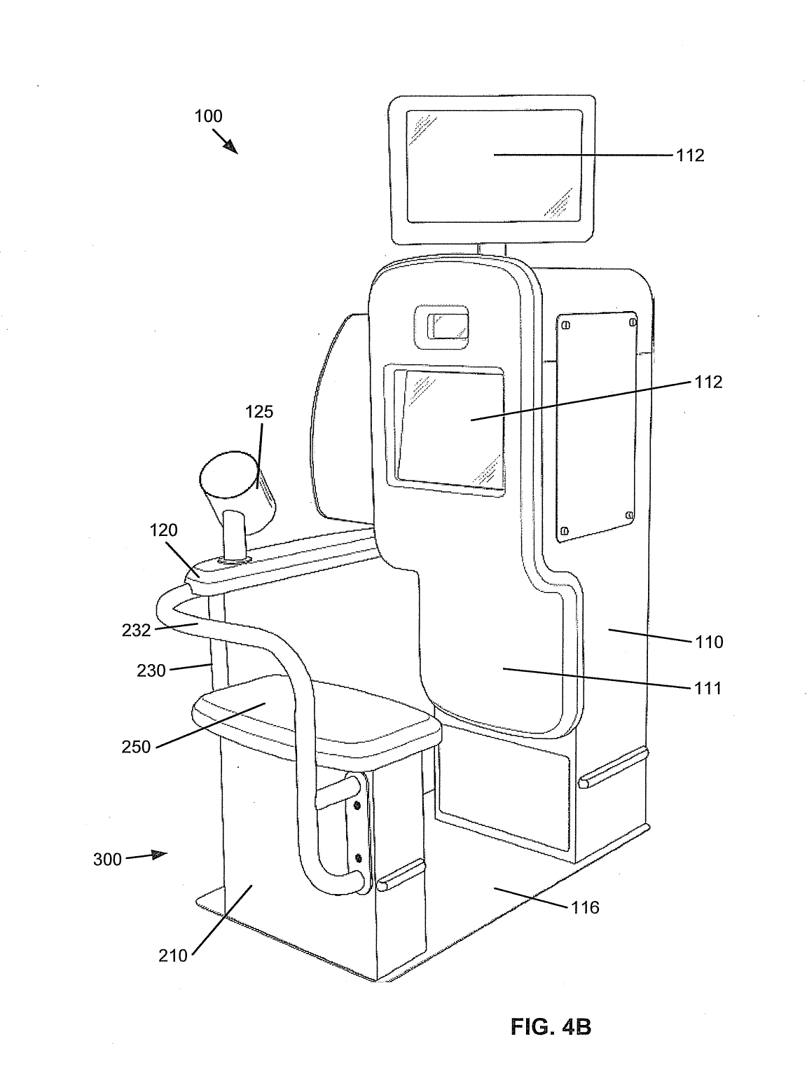

FIG. 4A is a perspective view of a kiosk 100 according to an embodiment of the invention. The kiosk 100 of FIG. 4A is similar to the kiosk 100 of FIGS. 1A-D, except that instead of the rotatable seat assembly 200/201 described in FIGS. 2A-H, the kiosk 100 of FIG. 4A may include a fixed seat assembly 300. FIG. 4B is a perspective view of a kiosk according to an embodiment of the invention. The kiosk 100 of FIG. 4B may also include a fixed seat assembly 300, and a bar 232 may also be provided. The bar 232 may be attached to the fixed seat assembly 300 and/or some other structure of the kiosk 100, such as the arm rest 120 and/or seat post 230. The bar 232 may be disposed near a side of the seat assembly 300 farthest from the front side 110 of the kiosk 100 at a position higher than a top surface of the seat 250, so that the user may be able to use the bar 232 as a back rest while facing the front side 110 of the kiosk 100. A user, for example a wheelchair-bound user, may also be able to use the bar 232 as an assist for getting onto the seat 250. Note that in some kiosk 100 embodiments with a fixed seat assembly 300 and a seat post 230, the seat post 230 may be attached to the seat support 210 instead of or in addition to a floor surface such as the base plate 116.

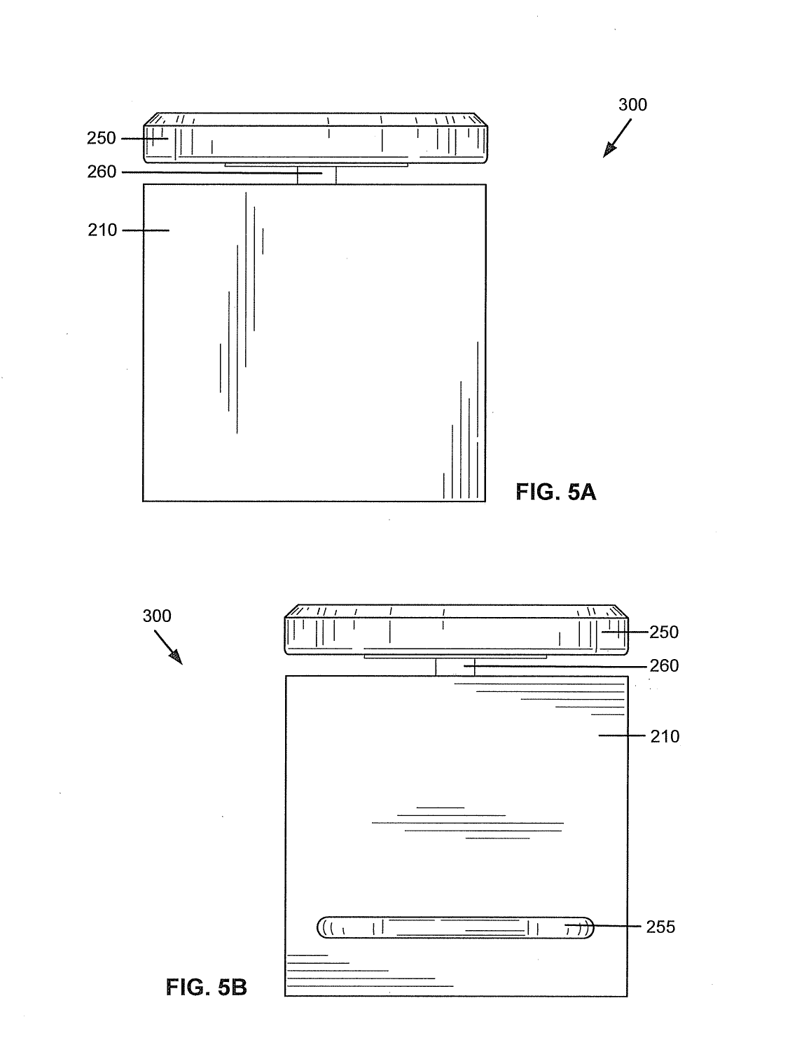

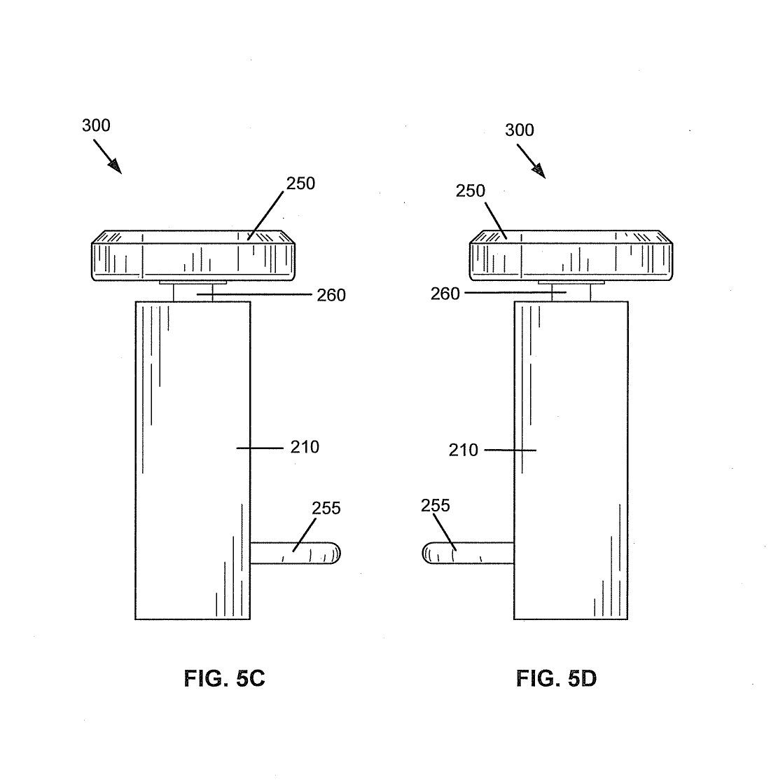

FIG. 5A-5D are elevation views of a fixed seat assembly 300 according to an embodiment of the invention. The fixed seat assembly 300 may be coupled to a seat post 230 or other element of the kiosk 100, or the fixed seat assembly 300 may be free standing. The fixed seat assembly 300 may include a seat support 210 and a seat 250. The seat 250 may be coupled to the seat support 210 with a load sensor 260.

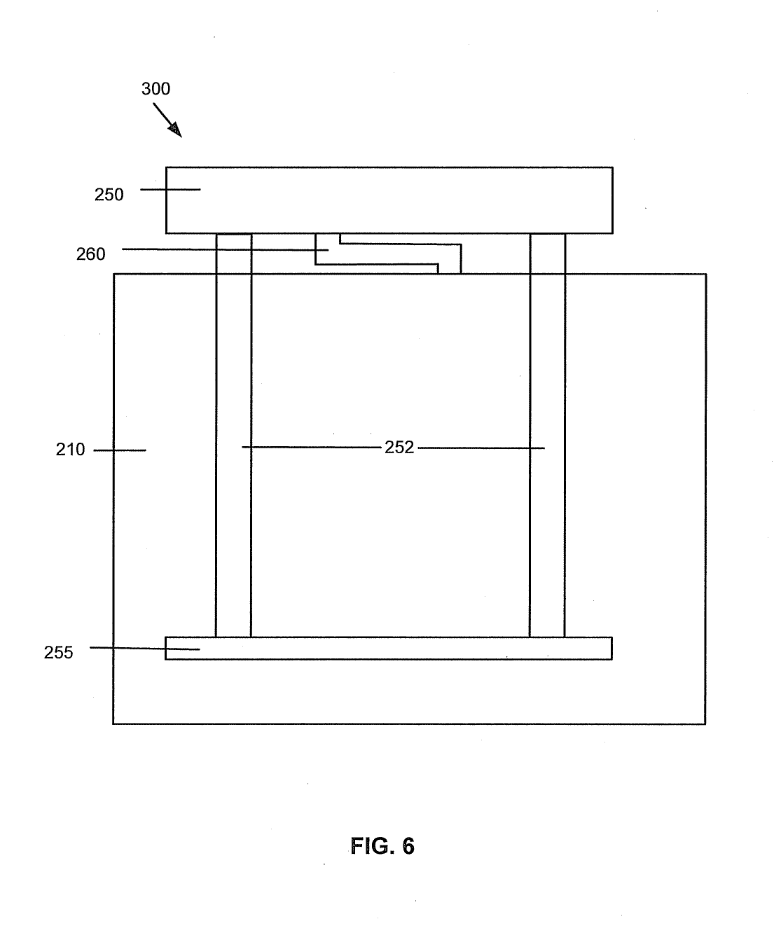

FIG. 6 is a cutaway front elevation view of the fixed seat assembly 300 according to an embodiment of the invention. Some embodiments of the seat assembly 300 may include a substantially hollow seat support 210 which may contain various components such as internal frame elements 280 and other components. For example, the load sensor 260 may be partially contained within the seat support 210. In one example, the load sensor 260 may be a load cell with one end attached to the seat 250 and another end attached to the seat support 210, although other load sensor types may be used instead of or in addition to the load cell. When a load, for example a user, is placed on the seat, the load sensor 260 may detect the load. For example, the load sensor may enable the computer of the kiosk 100 to determine and/or report a user's weight and/or body mass index. The foot rest 255 may be attached to the seat 250 so that the foot rest 255 is supported by the seat 250. Weight on the foot rest 255 may therefore be applied to the load sensor 260. For example, a user may place their feet on the foot rest 255 so that their entire weight may be measured by the load sensor 260. In some embodiments, such as the example of FIG. 6, a portion of the foot rest 255 may be contained within the seat support 210, with a portion of the foot rest 255 useful for supporting a user's feet protruding from an opening in the seat support 210. For example, the foot rest 255 may include vertical connecting elements 252 which may connect the foot rest 255 with the seat 250. These vertical connecting elements 252 may be inside the seat support 210. One or more bolts 290 may be disposed on the top of the seat support 210 and may extend to a position beneath the seat 250 without making contact with the seat 250. Therefore, if the load sensor 260 is deformed, for example because an excessive or unexpected load is applied to the seat 250, the weight of the seat 250 and the load on the seat 250 may be supported by the bolts 290 (and therefore the seat support 210).

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. Thus, the present embodiments should not be limited by any of the above-described embodiments.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the teens “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 212, paragraph 6. Claims that do not expressly include the phrase “means for” or “step for” are not to be interpreted under 35 U.S.C. 212, paragraph 6.

What is claimed is:

1. A kiosk comprising:
a main body comprising a front surface;
a vertical seat post located near the main body adjacent to the front surface of the main body; and
a seat assembly rotatably coupled to the seat post and configured to be rotated about the seat post between a position substantially parallel to the front surface of the main body and a position substantially perpendicular to the front surface of the main body.

2. The kiosk of claim 1, wherein the seat assembly comprises a caster disposed on a bottom side of the seat assembly and configured to contact a floor surface.

3. The kiosk of claim 1, further comprising an arm rest assembly connecting the main body to the seat post.

4. The kiosk of claim 1, wherein the main body further comprises:
a processor; and
a display in communication with the processor, the display disposed on the front surface of the main body.

5. The kiosk of claim 4, further comprising an arm rest assembly connecting the main body to the seat post, wherein the arm rest assembly comprises a blood pressure cuff in communication with the processor and configured to provide blood pressure data to the processor.

6. The kiosk of claim 4, wherein the seat assembly comprises:
a seat support rotatably coupled to the seat post;
a seat disposed on a top side of the seat support; and
a load sensor disposed between the seat support and the seat and connected to the seat support and the seat, the load sensor being configured to measure a load applied to the seat.

7. The kiosk of claim 6, wherein the load sensor is a load cell.

8. The kiosk of claim 6, wherein the seat assembly further comprises a foot rest supported by the seat so that weight on the foot rest is applied to the load sensor.

9. The kiosk of claim 8, wherein the foot rest is partially disposed within the seat support.

10. The kiosk of claim 6, wherein the seat box comprises a bolt protruding from the top side of the seat support and configured to support the seat when the load sensor is deformed.

11. The kiosk of claim 6, further comprising a data port in communication with the processor and disposed on the main body, the data port being configured to transmit data from the processor.

12. The kiosk of claim 6, further comprising a vision testing device in communication with the processor and configured to provide vision data to the processor.

13. The kiosk of claim 6, further comprising an input device in communication with the processor.

14. The kiosk of claim 13, wherein the input device comprises a button, a keyboard, a touch screen, a barcode scanner, and/or a magnetic card swipe device.

15. The kiosk of claim 10, wherein;
the load sensor is in communication with the processor and is configured to provide load data to the processor; and
the processor is configured to use the load data to calculate a weight and/or a body mass index.

16. The kiosk of claim 1, wherein the seat assembly comprises a latch mechanism configured to selectively hold the seat assembly in the position substantially parallel to the front surface of the main body and the position substantially perpendicular to the front surface of the main body.

17. The kiosk of claim 16, wherein the latch mechanism is partially disposed within the seat support.

18. The kiosk of claim 16, wherein the latch mechanism is configured to selectively substantially simultaneously engage the seat assembly with the seat post and to a floor surface.

19. The kiosk of claim 18, wherein:
the seat post comprises a plurality of holes; and
the latch mechanism comprises:
a post rod configured to selectively enter a first one of the plurality of holes in the seat post when the seat assembly is in the position substantially parallel to the front surface of the main body and to selectively enter a second one of the plurality of holes in the seat post when the seat assembly is in the position substantially perpendicular to the front surface of the main body; and
a floor rod configured to selectively enter a first hole in the floor surface when the seat assembly is in the position substantially parallel to the front surface of the main body and to selectively enter a second hole in the floor surface when the seat assembly is in the position substantially perpendicular to the front surface of the main body.

20. The kiosk of claim 19, wherein the post rod and the floor rod are mechanically linked so that the floor rod can only enter the first hole in the floor surface or the second hole in the floor surface when the post rod has entered one of the plurality of holes in the seat post.

21. A kiosk comprising:
a main body comprising a front surface; and
a seat assembly located near the main body adjacent to the front surface of the main body, the seat assembly comprising:
a seat support;
a seat disposed on a top side of the seat support; and
a load sensor disposed between the seat support and the seat and connected to the seat support and the seat, the load sensor being configured to measure a load applied to the seat.

22. The kiosk of claim 21, wherein the load sensor is a load cell.

23. The kiosk of claim 21, further comprising an arm rest assembly connecting the main body to the seat assembly.

24. The kiosk of claim 21, wherein the main body further comprises:
a processor; and
a display in communication with the processor, the display disposed on the front surface of the main body.

25. The kiosk of claim 24, further comprising an arm rest assembly connecting the main body to the seat assembly, wherein the arm rest assembly comprises a blood pressure cuff in communication with the processor and configured to provide blood pressure data to the processor.

26. The kiosk of claim 21, wherein the seat assembly further comprises a foot rest supported by the seat so that weight on the foot rest is applied to the load sensor.

27. The kiosk of claim 26, wherein the footrest is partially disposed within the seat support.

28. The kiosk of claim 21, wherein the seat support comprises a bolt protruding from the top side of the seat support and configured to support the seat when the load sensor is deformed.

29. The kiosk of claim 24, wherein;
the load sensor is in communication with the processor and is configured to provide load data to the processor; and
the processor is configured to use the load data to calculate a weight and/or a body mass index.

30. The kiosk of claim 24, further comprising a data port in communication with the processor and disposed on the main body, the data port being configured to transmit data from the processor.

31. The kiosk of claim 24, further comprising a vision testing device in communication with the processor and configured to provide vision data to the processor.

32. The kiosk of claim 24, further comprising an input device in communication with the processor.

33. The kiosk of claim 32; wherein the input device comprises a button, a keyboard, a touch screen, a barcode scanner, and/or a magnetic card swipe device.

34. The kiosk of claim 21, further comprising a bar disposed adjacent to the seat assembly, wherein at least a portion of the bar is at a position higher than the seat.
